(19) United States
(12) Patent Application Publication
(10) Pub. No.: US 2014/0114141 A1
(43) Pub. Date: Apr. 24, 2014

(54) SYSTEM FOR PROCESSING DECAYING PERIODIC PHYSIOLOGICAL SIGNALS

(71) Applicant: Wen Jea WHAN, El Monte, CA (US)

(72) Inventor: Wen Jea WHAN, El Monte, CA (US)

(21) Appl. No.: 14/146,282

(22) Filed: Jan. 2, 2014

Related U.S. Application Data
(63) Continuation-in-part of application No. 12/184,744, filed on Aug. 1, 2008, now abandoned.
Application No. 12/184,744 is a continuation-in-part of application No. 10/855,839, filed on May 28, 2004, now abandoned.

(51) Int. Cl. A61B 5/00; A61B 5/024; A61B 5/08; A61B 5/0402

(52) U.S. Cl. CPC A61B 5/7275; A61B 5/4836; A61B 5/7246; A61B 5/0402; A61B 5/7225; A61B 5/024; A61B 5/08
USPC 600/300

(57) ABSTRACT

A system for processing decaying periodic physiological signals comprises at least a sensor, a physiological signal-digitizing device, and a digital-signal processing unit to establish a parameterized database according to P^{j}ᵢ, S^{j}ᵢ, the relationship between P^{j}ᵢ and S^{j}ᵢ and the change with respect to time or locations of the sensors on the human body for evaluating the physiological or mental conditions. The system is applicable to processing and utilizing the decaying periodic physiological signals, such as heartbeats, respiration and ECG.

### CROSS-REFERENCE TO RELATED DOCUMENTS

The present invention is a continuation in part (CIP) to a U.S. patent application Ser. No. 12/184,744 entitled “SYSTEM FOR PROCESSING DECAYING PERIODIC PHYSIOLOGICAL SIGNALS” filed on Oct. 1, 2008; said U.S. patent application Ser. No. 12/184,744 is a continuation in part to a U.S. patent application Ser. No. 10/855,839 entitled “Method and system for processing periodic physiological signals” filed on May 28, 2004.

### BACKGROUND OF THE INVENTION

1. Field of the Invention

The present invention is related to a system utilizing the decaying characteristics of periodic physiological signals for decomposing the periodic decaying physiological signals of a human body numerically into series of exponential decays represented by poles P and residuals S as signatures of the physiological and mental conditions of the human body. The present invention is applicable to processing the decaying periodic physiological signals, such as heartbeat, respiration and ECG of a human body.

2. Description of the Prior Art

A healthy human body will naturally have some physiological phenomena, e.g. heartbeat, respiration and pulses. Long ago, doctors have evaluated health conditions of a person by the symptoms of these physiological phenomena to prevent and cure the disease. Generally, the practitioner of Chinese medicine by feeling the pulse and the doctor practicing western medicine by stethoscopes or hemadynamometers observe the change of heartbeat, pulses and respiration. In this modern time, doctors could collect these periodic signals by electronic devices for primary evaluation.

Taking heartbeat as an example, the heartbeat of a human body has the feature of regular pattern and repeatedly appearing. The status of these physiological signals can be measured by sonography or electrocardiography (ECG). Referring to FIG. 1, a standard ECG consisting of a serial wave group is taken as an instance. Each wave group representing a heartbeat cycle includes the P-wave, QRS complex, T-wave and U-wave, wherein:

(1) the P-wave is generated by depolarizing the atrium and is the first wave of the periodic wave group and shows the depolarization procedure of left and right atrium. In addition, the individual periodic waveform has the characteristics of decaying.

(2) The QRS complex includes three waves connected closely, wherein the Q-wave is the first downward wave, the R-wave is an upright high-tip wave after the Q-wave, and the S-wave is downward after the R-wave. In case the three waves are connected closely, they are called as QRS complex and reflect the procedure of depolarizing of left and right ventricles.

(3) The T-wave located behind the ST segment is a low and long-duration wave and is generated by ventricular repolarization.

(4) The U-wave located after the T-wave is lower and smaller and is thought to relate to after-depolarization, which interrupts or follows repolarization. Usually the U-wave is neglected in the ECG waveforms.

Generally the ECG waveforms of a normal human body have clear and complete wave change. In case the body has abnormal conditions or suffers stimulations, the ECG will show a waveform that is different from the normal waveforms and can be used to evaluate the health conditions of human body. However, any figures of physiological signals have to be judged by a doctor with professional training to have a result. If one can know the health conditions without the evaluation by the doctor, time will be saved. Further, the physiological conditions of a person will depend on mental conditions, such as excitement, nervousness, or fear. The body will have the phenomena of increasing heartbeat rate, sweating, and vasoconstriction. It is obvious that the mental conditions can be evaluated by observing any physiological conditions. Further more, every repeating signal decays with respect to time and could be decomposed numerically into poles and residuals. These parameters are proposed to be used as a signature for the physical and mental condition of a human. Similar technologies had been proposed as an advanced weapon detection system which applies the decay for the stimulated eddy current as the signature for different metal weapons (U.S. Pat. No. 6,469,624 on “NON-OBTRUSIVE WEAPON DETECTION SYSTEM”, Inventors: Dr. Wen J. Whan and Dr. George V. Keller; U.S. Pat. No. 5,552,705 on “NON-OBTRUSIVE WEAPON DETECTION SYSTEM AND METHOD FOR DISCRIMINATING BETWEEN A CONCEALED WEAPON AND OTHER METAL OBJECTS”, Inventor: Dr. George V. Keller.)

In view of this, the inventor provides a system for processing physiological signals, which takes advantage of characteristics of time decay and periodic performance of the periodic physiological signals to obtain parameters (poles and residual) as signatures of the diseases or abnormal mental conditions.

### SUMMARY OF THE INVENTION

The object of the present invention is to provide a system for processing decaying periodic physiological signals to receive series of physiological signals with respect to time or different sensor locations and to provide an evaluating result via the comparison with a parameterized database to evaluate the physiological and mental conditions of the human body.

To achieve the above objects, a system for processing decaying periodic physiological signals according to the present invention is provided and comprises at least a sensor for sensing the periodic physiological signals of a human body and outputting the physiological signals, a physiological signal-digitizing device for receiving and digitizing the physiological signals to form digitized decaying periodic physiological signals D, and a digital-signal processing unit for decomposing the digitized decaying periodic signals D of the human body into poles P and residuals S. A parameterized database is then established according to P^{j}ᵢ, S^{j}ᵢ, the relationship between P^{j}ᵢ and S^{j}ᵢ and the change with respect to time or space (locations), and outputting an evaluating result by comparing the parameterized database with original data value to judge the physiological or mental conditions of the human body.

The present invention will be apparent after reading the detailed description of the preferred embodiments thereof in reference to the accompanying drawings.

### BRIEF DESCRIPTION OF THE DRAWINGS

FIG. 1 is a schematic diagram of traditional standard decaying periodic waveforms of ECG;

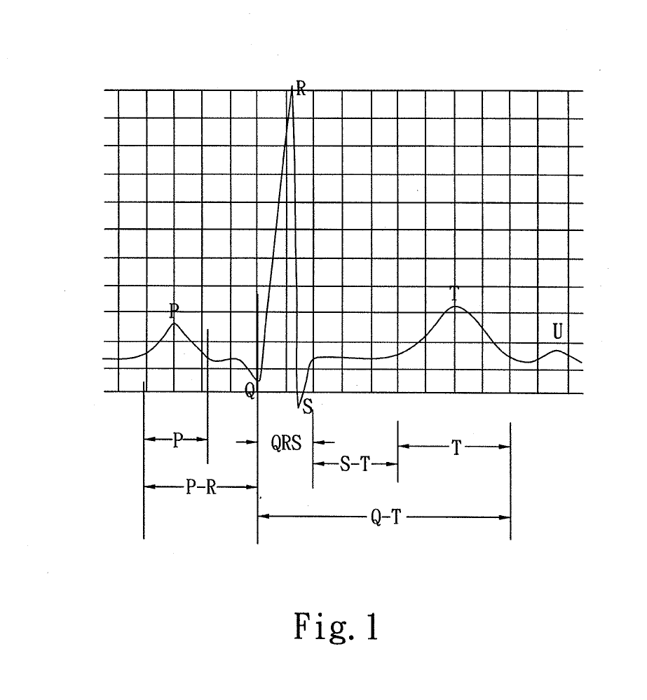

FIG. 2 is a schematic view of a system for processing decaying periodic physiological signals of the present invention;

FIG. 3 is a decaying periodic signal waveform diagram recorded from an ultrasonic detector measuring the heartbeat;

FIG. 4 is a decaying periodic signal waveform diagram recording the relative flow rate of a blood vessel of a thumb at different temperatures by a laser Doppler flowmeter;

FIG. 5 is a three dimension waveform diagram of electromagnetic decaying signals measured by eight receiving coils on the vertical position of the main blood vessel, which is exerted by external periodic electromagnetic waves; and

FIG. 6 is a schematic view of an embodiment of the present invention of the poles P^{j}ᵢ, S^{j}ᵢ and evaluating results, where the poles are decomposed from the signals of FIG. 5.

### DETAILED DESCRIPTION OF THE PREFERRED EMBODIMENT

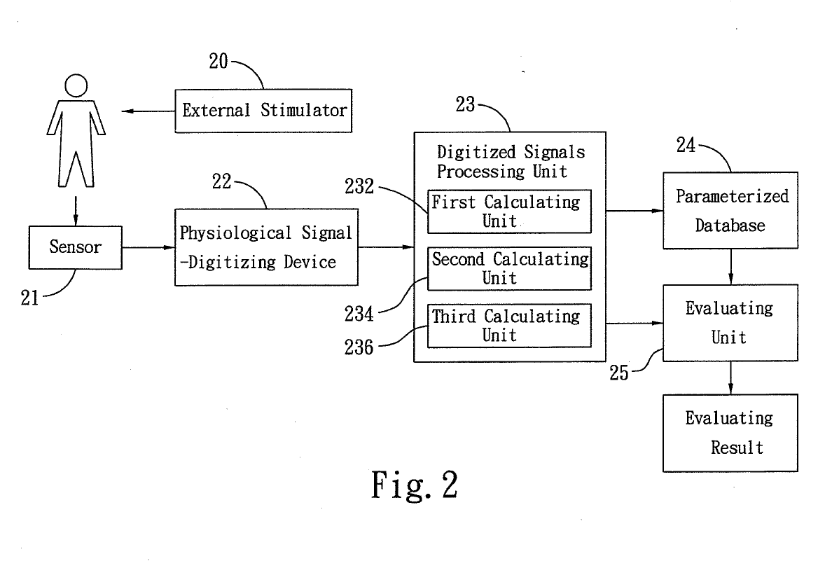

The system for processing decaying periodic physiological signals is depicted in FIG. 2. The system includes at least a sensor 21 for sensing the periodic decaying periodic physiological signals of a human body, a physiological signal-digitizing device 22 for receiving and digitizing the physiological signals to form digitized decaying periodic physiological signals D, and a digital-signal processing unit 23 for decomposing the digitized decaying periodic physiological signals D of the human body into poles P and residuals S, wherein the P represents the decay rate of signals, the S represents the contribution value of the P. By taking j as the serial number of signals, the D^{j} is periodic signals with different characters, or the same periodic signals in different measuring time or locations. The D^{j}, P and S satisfy the following formula:

$${D}^{j} = {\sum }_{i = 1}^{m} {S}_{i}^{j} {e}^{- {P}_{i}^{j}}$$

Wherein m represents the maximum number of poles from the decaying signals to be decomposed and is collected from a human body. The value of P^{j}ᵢ, or S^{j}ᵢ can be used as the signatures of the physiological or mental conditions of said human body.

The parameterized database 24 is established according to P^{j}ᵢ, S^{j}ᵢ, from different human bodies, the relationship between P^{j}ᵢ and S^{j}ᵢ and their change with respect to time or space (locations) by the numerical method.

The digital signals processing unit 23 includes a first, a second, and a third calculating unit 232, 234, 236. The first calculating unit 232 is used for calculating ratios or statistic patterns between P^{j}ᵢ and S^{j}ᵢ that can be used as signatures of the physiological or mental conditions of said human body. The second calculating unit 234 is used for calculating the change with respect to time or locations for P^{j}ᵢ or S^{j}ᵢ that can be used as signatures of the physiological or mental conditions of said human body. The third calculating unit 236 is used for calculating the change with respect to time or locations for ratio and statistical patterns of P^{j}ᵢ and S^{j}ᵢ that also can be used as signatures of the physiological or mental conditions of said human body.

The system is also provided with an external stimulator 20 for stimulating the human body to generate decaying periodic physiological signals or change the human body's natural decaying periodic physiological signals. In other words, the decaying periodic physiological signals are obtained from said human body exerted by the external stimulator 20 and then the decaying periodic physiological signals can be collected and digitized. The external stimulation generated by said external stimulator 20 includes voltage, electromagnetic waves, ultrasonic waves, heat, or pressure.

Moreover, the system further comprises a comparison unit 25 and an evaluation unit 27. As depicted in FIG. 2, the comparison unit 25 is used to compare the values of the poles P^{j}ᵢ and residuals S^{j} from a certain human body with those stored in the parameterized database and output a comparison result 26. The comparison result 26 is received by the evaluation unit 27. As a result, the evaluating unit 27 would output an evaluation result 28 representing the physiological or mental condition of that human body according to the received comparison result 26.

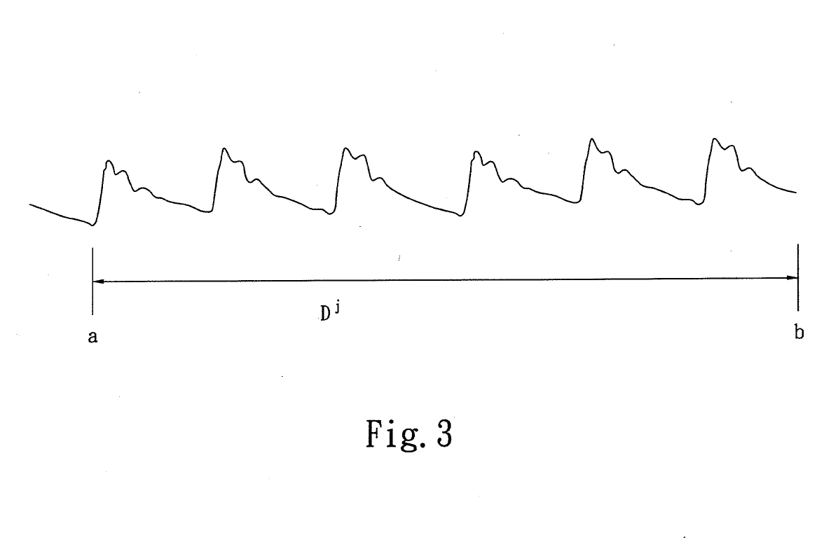

Then take a specific physiological signal as an embodiment of the present invention. Use an ultrasonic detector as the sensor 21 to measure a subject's heartbeats to obtain a heartbeat ultrasonic waveform diagram as shown in FIG. 3. Setting the range of physiological signals from point (a) to point (b), which shows five decaying signals and transferring the signals between these two points to physiological signal-digitizing device 22 to obtain digitized decaying periodic physiological signals D. Then transferring the digitized signals to digital-signal processing unit 23 to decompose D into the poles P and the residuals S by the numerical method and the D^{j}, P and S can satisfy the following formula:

$${D}^{j} = {\sum }_{i = 1}^{m} {S}_{i}^{j} {e}^{- {P}_{i}^{j}}$$

Wherein m represents the maximum number of poles from the decaying signals to be decomposed and is collected from a human body. The P value is larger; the decreasing rate is faster. Further calculating the relationship between the P^{j}ᵢ and S^{j}ᵢ or the relationship between the P^{j}ᵢ and S^{j}ᵢ and the change with respect to time/locations by a calculating unit and comparing those with a pre-determined parameterized database 24 by the comparison unit 25 to output a comparison result 26 indicating the physiological or mental conditions of a human body to show the subject's health conditions.

If the parameterized database hasn't been established, following the above-mentioned method to measure enough healthy subjects in statistics and analyzing the physiological signals of the subjects to establish a parameterized database 24. Store the database 24 in the system of the present invention for the reference of evaluating the healthy conditions. The database 24 can also be input and modified with parameterized data, e.g. gender, heights or weight to raise the accuracy of the database 24.

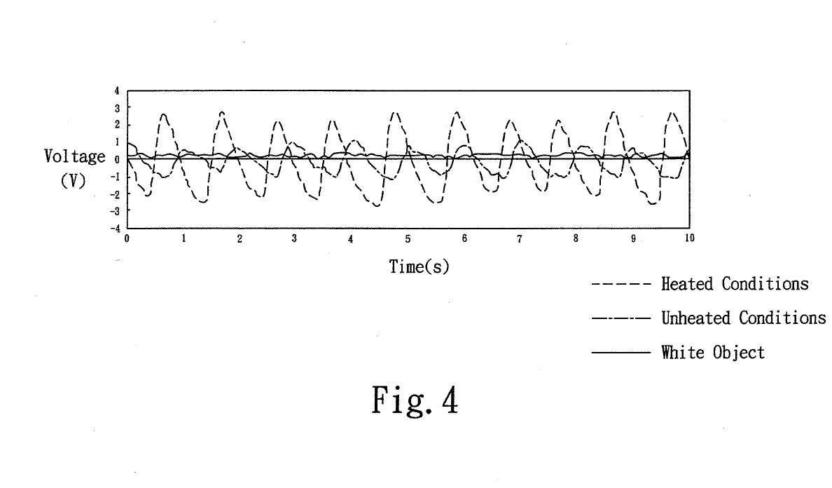

Besides, the system includes physiological signals of different measuring conditions. For example, taking laser Doppler flowmeter as a sensor 21 can measure the relative flow rate of the blood vessel of a thumb at different temperatures and obtain a diagram shown in FIG. 4, wherein the control is a white object. Measuring the physiological signals in unheated conditions and in heated conditions respectively and applying the digital-signal processing unit 23 to analyze and resort the signals to further establish the physiological reference data in different measuring conditions.

The present invention is suitable for evaluating physiological signals with different characters. For example, taking the electrocardiograph as a sensor 21 of the system to detect heartbeat of a human body, and applying the system mentioned above to digitize and decompose the physiological signals to store the P^{j}ᵢ, S^{j}ᵢ and time/locations in the digital-signal processing unit 23 and compare those with the ultrasonic data of heartbeats mentioned above to further obtain the physiological and mental conditions of a human body.

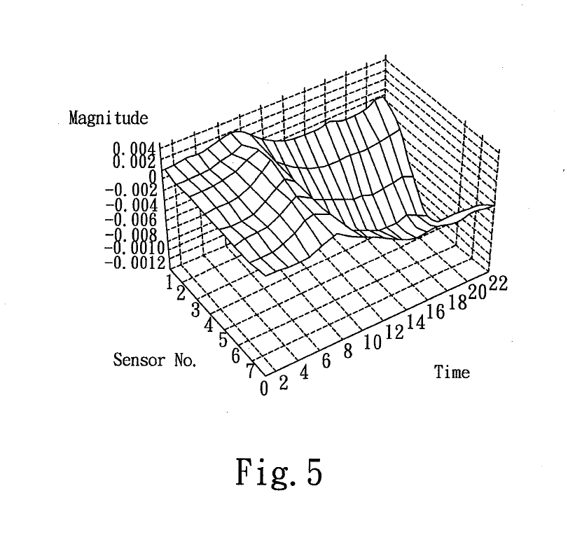

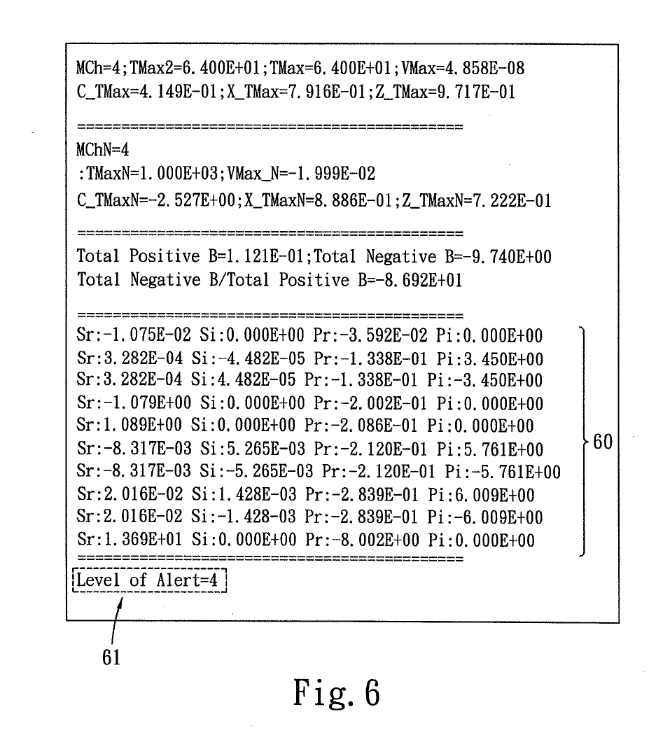

The present invention not only measure the physiological signals from a human body under normal conditions, but also can evaluate the health conditions when a human body is exerted by an external stimulation from the external stimulator 20. The external stimulation can generate decaying periodic physiological signals or change the natural decaying periodic physiological signals. The external stimulation can be voltage, electromagnetic waves, ultrasonic waves, heat, pressure, acupuncture, etc. For example, by stimulating external periodic stepwise electromagnetic waves, the main blood vessel is positioned with eight receiving coils. The decaying signals of electromagnetic field generated by Eddy current are shown in FIG. 5. The poles P^{j}ᵢ and residuals S^{j}ᵢ decomposed from the signals D are shown in FIG. 6 and labeled as 60. Then comparing these values with the pre-determined parameterized database 24 could obtain an evaluating result of physiological or mental conditions of a human body. The evaluation result shown in FIG. 6 and labeled as 61 can indicate the health level of the tested person.

The foregoing description of the preferred embodiments of this invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of this invention.

Thus, the present invention has following advantages:
1. The present invention not only can be used as analysis of single physiological signals, but also can analyze composite physiological signals to raise the accuracy of evaluation.
2. By using the present invention to evaluate body conditions, it is controlled by electronic devices without judging by a doctor and is convenient for operating to figure out the health conditions.
3. Because the different measuring conditions will result in different conclusions, operators can input parameters with different terms, e.g. heights, weights, external stimulations or temperatures, and then the parameterized database can make the results more accurate.

The system for processing decaying periodic physiological signals not only offers the evaluation of physiological conditions but also offers the evaluation of mental conditions. The practitioner of Chinese medicine by feeling the pulse and the doctor practicing western medicine by stethoscopes or hemadynamometers observe the change of heartbeat, pulses and respiration. In this modern time, doctors could collect these periodic signals by electronic devices for primary evaluation. By using this invention, the changes of the decaying periodic signals will be presented in a scientific and therefore more accurate way.

As stated in the above disclosed, the present invention can surely achieve its expected objects to provide a system for processing decaying periodic physiological signals for the public or the medical personnel to evaluate the health conditions. It has its industrial practical value.

1. A system for processing decaying periodic physiological signals including:
at least a sensor, for sensing decaying periodic physiological signals from a human body;
a physiological signal-digitizing device, for receiving and digitizing the decaying periodic physiological signals to form digitized decaying periodic physiological signals D; and
a digital-signal processing unit, for decomposing the digitized decaying periodic physiological signals D into poles P and residuals S, wherein the poles P represent the decay rate of signals, the residuals S represent the contribution value of the P, and by taking j as the serial number of signals, D^{j} represent different signals or the same signal measured at different time points or locations, and D^{j}, P and S satisfy the following formula:
$${D}^{j} = {\sum }_{i = 1}^{m} {S}_{i}^{j} {e}^{- {P}_{i}^{j}}$$
wherein m represents the maximum number of poles from the decaying periodic physiological signals to be decomposed and is collected from a human body, P^{j}ᵢ represents the i^{th} pole of the j^{th} periodic human signals or j^{th} location of the sensor, and S^{j}ᵢ represents the contribution value or residual value of the pole; thus, obtaining the values of the poles P^{j}ᵢ and residuals S^{j}ᵢ by decomposing the decaying periodic physiological signals by using a numerical method.

2. The system for processing decaying periodic physiological signals according to claim 1, further comprising an external stimulator for stimulating the human body to generate decaying periodic physiological signals or change the human body's natural decaying periodic physiological signals, wherein the decaying periodic physiological signals obtained from said human body exerted by the external stimulator are digitized by the physiological signal-digitizing device.

3. The system for processing decaying periodic physiological signals according to claim 2, further comprising:
a parameterized database, established according to the values P^{j}ᵢ, S^{j}ᵢ from different human bodies, the relationship between the P^{j}ᵢ and S^{j}ᵢ and their change with respect to time or locations by the numerical method; and
a comparison unit, used for comparing the values of the poles P^{j}ᵢ and residuals S^{j} from the human body with the data in the parameterized database and outputting a comparison result.

4. The system for processing decaying periodic physiological signals according to claim 3, wherein the digital-signal processing unit further comprises a first calculating unit for calculating ratios or statistic patterns between P^{j}ᵢ and S^{j}ᵢ.

5. The system for processing decaying periodic physiological signals according to claim 3, wherein the digital-signal processing unit further comprises a second calculating unit for calculating the change with respect to time or locations for P^{j}ᵢ or S^{j}ᵢ.

6. The system for processing decaying periodic physiological signals according to claim 3, wherein the digital-signal processing unit further comprises a third calculating unit for calculating the change with respect to time or locations for ratio and statistical patterns of P^{j}ᵢ and S^{j}ᵢ.

7. The system for processing decaying periodic physiological signals according to claim 2, further comprising:
an evaluating unit, used for outputting an evaluating result according to the comparison result from the comparison unit.

8. The system for processing decaying periodic physiological signals according to claim 1, further comprising:
a parameterized database, established according to the values P^{j}ᵢ, S^{j}ᵢ from different human bodies, the relationship between the P^{j}ᵢ and S^{j}ᵢ and their change with respect to time or locations by the numerical method; and
a comparison unit, used for comparing the values of the poles P^{j}ᵢ and residuals S^{j} from the human body with the data in the parameterized database and outputting a comparison result.

9. The system for processing decaying periodic physiological signals according to claim 8, wherein the digital-signal processing unit further comprises a first calculating unit for calculating ratios or statistic patterns between P^{j}ᵢ and S^{j}ᵢ.

10. The system for processing decaying periodic physiological signals according to claim 8, wherein the digital-signal processing unit further comprises a second calculating unit for calculating the change with respect to time or locations for P^{j}ᵢ or S^{j}ᵢ.

11. The system for processing decaying periodic physiological signals according to claim 8, wherein the digital-signal processing unit further comprises a third calculating unit for calculating the change with respect to time or locations for ratio and statistical patterns of and S^{j}ᵢ.

12. The system for processing decaying periodic physiological signals according to claim 1, further comprising:
an evaluating unit, for outputting an evaluating result according to the comparison result from the comparison unit.
